(19) United States
(12) Patent Application Publication
(10) Pub. No.: US 2008/0058865 A1
(43) Pub. Date: Mar. 6, 2008

(54) Surgical closure device and associated method

(76) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(21) Appl. No.: 11/894,669

(22) Filed: Aug. 21, 2007

Related U.S. Application Data
(60) Provisional application No. 60/839,144, filed on Aug. 21, 2006.

(51) Int. Cl. A61B 17/08

(52) U.S. Cl. 606/215; 606/213

(57) ABSTRACT

A surgical closure method utilizes a surgical closure device having an elongate tubular shaft, an elongate rod extending longitudinally through the shaft, and a disk attached to a distal end of the rod. The method comprises (a) inserting a distal end portion of the shaft into a patient, (b) thereafter shifting the rod relative to the shaft so that the disk is located distally of a distal end of the shaft, (c) manipulating the rod to insert the disk through a hole in a wall of a hollow internal organ of the patient, (e) pulling the rod in a proximal direction to bring the inserted disk into contact with the organ wall, (f) thereafter ejecting a plurality of fasteners from the distal end of the shaft through the wall and at least partially through the disk while the disk is maintained in contact with the wall, and (g) after the ejecting of the fasteners, separating the disk from the rod, thereby completing a closure of the hole.

### CROSS-REFERENCE TO RELATED APPLICATION

This application claims the benefit of U.S. Provisional Patent Application No. 60/839,144 filed Aug. 21, 2006.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduce trauma to the individual even more than laparoscopic procedures. Hospital convalescence stays are even shorter. There are some potential problems with the procedures, such as the difficulty in forming a fluid tight closure of the perforation formed in the wall of the hollow internal body organ.

### OBJECTS OF THE INVENTION

It is an object of the present invention to provide improvements on the afore-described surgical procedures.

It is a more specific object of the present invention to provide a method for closing a perforation or opening in an organ wall, particularly where the organ wall is located in the abdominal cavity or other body space.

A further object of the present invention is to provide a device and/or a surgical kit useful in performing the closure method.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A surgical closure device in accordance with the present invention comprises an elongate tubular shaft, an elongate rod extending longitudinally through the shaft, a disk attached to a distal end of the rod, a plurality of fasteners carried by the shaft at a distal end thereof, and an ejection mechanism engageable with the fasteners for ejecting the fasteners at least partially through the disk, the disk being separable from the rod.

The fasteners may take the form of barbed pins or tacks. Alternatively, the fasteners may be staples. In the latter dace, the disk may be attached on a distal side to an anvil element, the anvil element and the disk being separable as a single unit from the rod. The anvil element is also pivotably attached to the rod.

It is specifically contemplated that the disk is circular and that the fasteners are disposed in a circular array and insert into the disk about an outer edge thereof. The disk is pivotably attached to the rod at a center of the disk.

The present invention finds particular application in transorgan surgery. In such applications, the shaft and the rod are elongate and flexible.

In a particular embodiment of the present invention, a ring is disposed on the shaft at the distal end thereof proximally of the disk, the ring is attachable to the disk by means of the fasteners.

In an alternative embodiment of the present invention, the disk is a first disk and the surgical closure device further comprises a second disk disposed on the shaft at the distal end thereof proximally of the first disk. The second disk is attachable to the first disk by means of the fasteners.

Pursuant to another feature of the present invention, the disk is pivotably attached to the distal end of the rod. The surgical closure device may further comprise a biasing element engageable with the disk for tilting the disk relative to the rod so that the disk is disposed at least partially parallel to a distal end portion of the rod. The biasing element may incorporate a spring.

In performing a surgical closure method in accordance with the present invention, one provides a surgical closure device having an elongate tubular shaft, an elongate rod extending longitudinally through the shaft, and a disk attached to a distal end of the rod. The method further comprises (a) inserting a distal end portion of the shaft into a patient, (b) thereafter shifting the rod relative to the shaft so that the disk is located distally of a distal end of the shaft, (c) manipulating the rod to insert the disk through a hole in a wall of a hollow internal organ of the patient, (e) pulling the rod in a proximal direction to bring the inserted disk into contact with the organ wall, (f) thereafter ejecting a plurality of fasteners from the distal end of the shaft through the wall and at least partially through the disk while the disk is maintained in contact with the wall, and (g) after the ejecting of the fasteners, separating the disk from the rod, thereby completing a closure of the hole.

The fasteners may include barbed elements.

In a preferred embodiment of the invention, the disk is pivotably attached to the distal end of the rod. The method then additionally comprises tilting the disk relative to the rod after the shifting of the rod in a distal direction relative to the shaft. The disk is inserted in the tilted orientation through the hole in the organ wall. The tilted orientation facilitates the passage of the disk through the hole in the organ wall.

The disk may be attached on a distal side to an anvil element. Where the disk is pivotably attached to the rod, the anvil element is also pivotably attached to the rod. In that case, the tilting of the disk includes a tilting of the anvil element, while the inserting of the tilted disk through the hole includes inserting the tilted anvil element through the hole. Also the method further comprises deforming the fasteners against the anvil during the ejecting of the fasteners from the distal end of the shaft, the separating of the disk from the rod including a separating of the anvil element from the rod, the anvil element and the disk being separated as a single unit from the rod.

The ejecting of the fasteners preferably includes ejecting the fasteners in a circular array about an outer edge of the disk.

Where the shaft and the rod are elongate and flexible, the inserting of a distal end portion of the shaft includes moving the distal end portion of the shaft along a curvilinear path, for instance, a path through a portion of the digestive system of the patient.

The method may further comprise placing a ring against the wall after the inserting of the disk through the hole. The placed ring is aligned with an edge of the disk and disposed on a side of the wall opposite the disk. The ejecting of the fasteners includes moving the fasteners through the ring and results in an attachment of the ring to the disk.

Alternatively, the method may further comprise placing a second disk against the wall after the inserting of the first disk through the hole. The placed second disk is aligned with an edge of the first disk and disposed on a side of the wall opposite the first disk. The ejecting of the fasteners includes moving the fasteners through the second disk and results in an attachment of the second disk to the first disk.

### BRIEF DESCRIPTION OF THE DRAWINGS

FIG. 1 is a schematic cross-sectional view of a person's upper digestive tract, showing a step in a surgical closure procedure in accordance with the present invention.

FIG. 2 is a schematic partial cross-sectional view of a person's stomach, showing disposition of a closure instrument in a subsequent step in the surgical closure procedure of FIG. 1.

FIG. 3 is a schematic partial perspective view of a modified distal end of the closure device of FIG. 2.

FIG. 4 is a schematic partial cross-sectional view similar to FIG. 2, depicting a later step in the surgical closure procedure.

FIG. 5 is a schematic partial cross-sectional view similar to FIGS. 2 and 4, depicting a further step in the surgical closure procedure.

FIG. 6 is a schematic partial cross-sectional view similar to FIGS. 2, 4 and 5, depicting an additional step in the surgical closure procedure.

FIG. 7 is a schematic partial cross-sectional view similar to FIGS. 2 and 4-6, depicting a yet another step in the surgical closure procedure.

FIG. 8 is a schematic perspective view of a hole in a stomach wall with a surgical closure device attached to the wall by the method shown in FIGS. 1-7, in accordance with the present invention.

FIG. 9 is a schematic cross-sectional view, taken along line IX-IX in FIG. 8.

FIG. 10 is a schematic perspective view of a hole in a stomach wall with another surgical closure device attached to the wall by a method as shown in FIGS. 1-7, in accordance with the present invention.

FIG. 11 is a schematic cross-sectional view, taken along line XI-XI in FIG. 10.

FIG. 12 is a schematic perspective view of a hole in a stomach wall with yet another surgical closure device attached to the wall by a method as shown in FIGS. 1-7, in accordance with the present invention.

FIG. 13 is a schematic perspective view of a hole in a stomach wall with yet a further surgical closure device attached to the wall by a method as shown in FIGS. 1-7, in accordance with the present invention.

FIG. 14 is a schematic partial side elevational view of a closure instrument utilizable in a method in accordance with the present invention, to install the closure device of FIG. 12 or 13.

FIG. 15 is a schematic perspective view, on an enlarged scale, depicting a surgical closure device releasably coupled to a distal end of a closure instrument in accordance with the present invention.

### DETAILED DESCRIPTION OF THE INVENTION

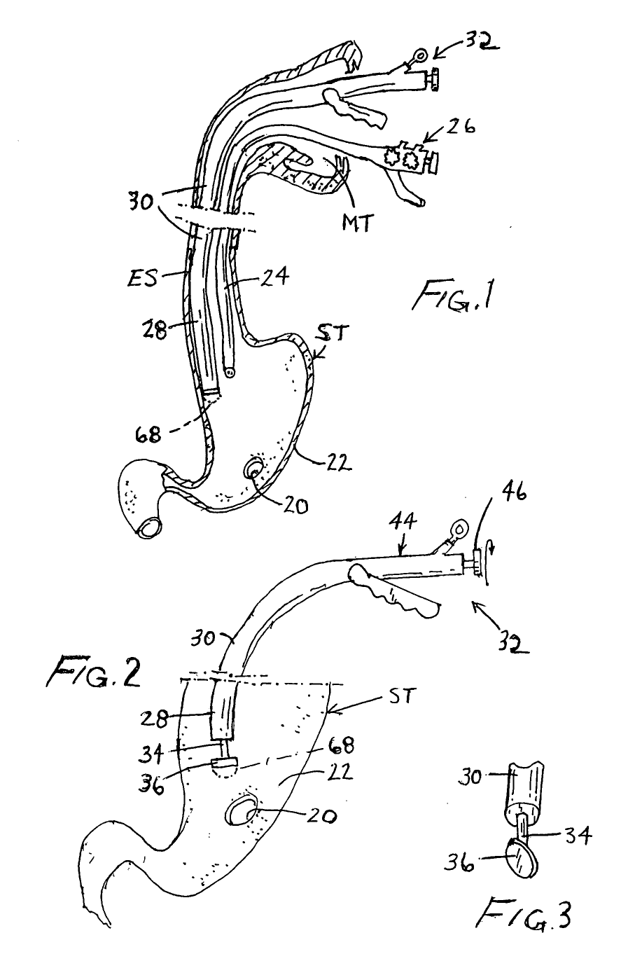

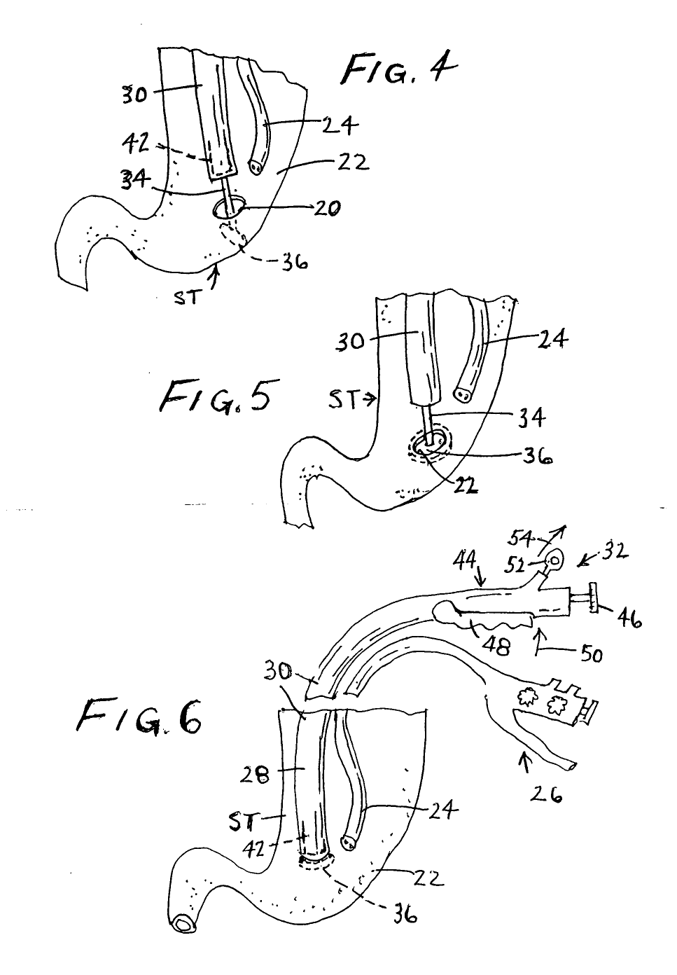

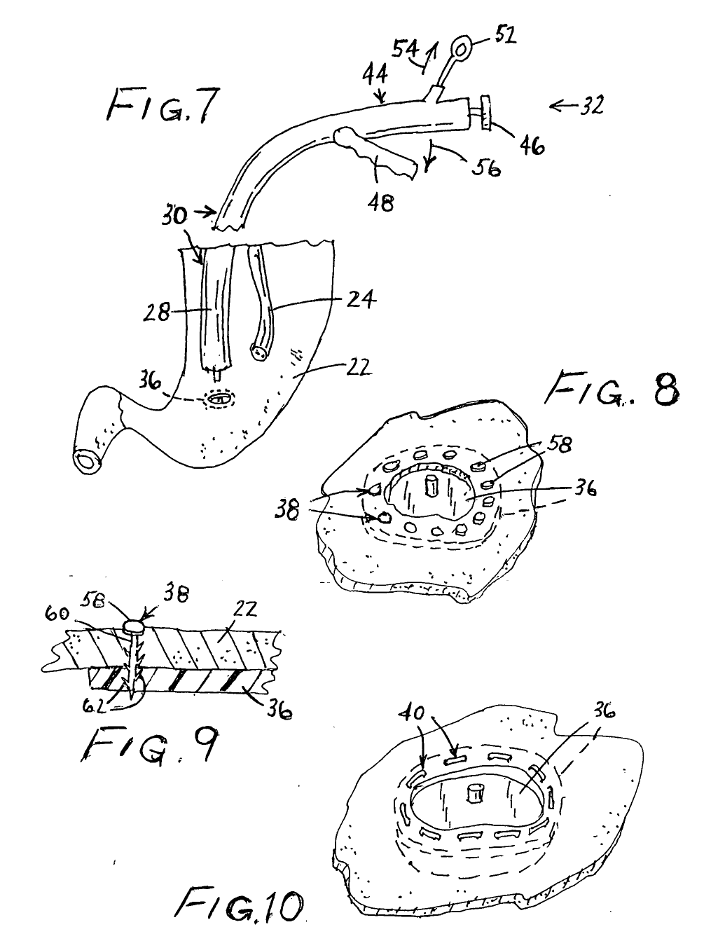

FIG. 1 depicts an initial step in a minimally invasive surgical operation for closing an opening or hole 20 formed in a wall 22 of an internal hollow body organ such as the stomach ST during a trans-organ surgical procedure as described in U.S. Pat. Nos. 5,297,536 and 5,458,131. A distal end portion 24 of an endoscope 26 is inserted into a patient PT via the patient's mouth MT and esophagus ES. A distal end potion 28 of a flexible tubular shaft 30 of a surgical closure device 32 is likewise inserted into the stomach ST.

Closure device 32 is similar in certain respects to a conventional EEA (end-to-end anastomosis) device. Differences between closure device 32 and an EEA device arise from the difference in function: an EEA device connects free ends of two tubular organs to one another to form a single lumen organ.

In addition to shaft 30, closure device 32 comprises an elongate flexible rod 34 (FIGS. 2, 3) extending longitudinally through the shaft, a disk 36 separably attached to a distal end of the rod, and a plurality of fasteners 38 or 40 (FIG. 8 or 10) carried by the shaft at a distal end thereof. An ejection mechanism 42 (FIG. 4, 15 . . . ) operatively mounted at the distal end of shaft 30 is engageable with fasteners 38 or 40 for ejecting the fasteners at least partially through disk 36.

As shown in FIG. 1, disk 36 is positioned flush against the distal end of shaft 30 during insertion of distal shaft portion 28 into the patient PT. Subsequently, under visual feedback provided by endoscope 26, a surgeon or endoscopist manipulates a handle 44 of closure device 32 and particularly a rotary knob 46 to cause rod 34 to advance in the proximal direction, thereby spacing disc 36 from the distal end of shaft 30 (FIG. 2). As shown in FIG. 3, disk 36 may be pivotably attached to the distal end of rod 34, so that upon a distal shifting of disk 36 relative to shaft 30, disk 36 may be oriented at an acute angle relative to rod 34 and shaft 40. The tilting of disk 36 facilitates an insertion of disk 36 through opening 20, as shown in FIG. 4. The passing of disk 36 through opening 20 may be accomplished by shifting the entire closure device 32 further into the patient, under visual guidance provided by endoscope 26.

Upon the passage of disk 36 through opening 20, closure device 32 is pulled back in the proximal direction until disk 36 is flush against stomach wall 22, as shown in FIG. 5. During this pulling back of closure device 32, disk 36 engages wall 22 and tilts as necessary to engage wall 22 all around opening 22. As in an EEA device, a turning of knob 46 shifts rod 34 in a proximal direction relative to shaft 30, so that wall 22 about opening 20 may be sandwiched between disk 36 and the distal end of shaft 30 (FIG. 6). Then, a handgrip actuator 48 is squeezed and pivoted, as indicated by an arrow 50 in FIG. 6, to operate ejection mechanism 42 to eject fasteners 38 or 40 through stomach wall 22 and at least partially through disk 36, thereby locking disk 36 against an outer surface of wall 22 and closing opening 20. Subsequently, an actuator or control 52 is manipulated, for instance, pulled in a proximal direction as indicated by an arrow 54 in FIGS. 6 and 7, to release or enable the separation of disk 36 from rod 34 (FIG. 7). Handgrip actuator 48 is returned to its original position, as indicated by an arrow 56 in FIG. 7, to retract or deactivate ejection mechanism 42.

The entire surgical closure procedure described above is performed under visual inspection afforded by endoscope 26, upon the separation of disk 36 from shaft 30, closure device 32 and endoscope 26 are withdrawn from the patient.

As shown in FIGS. 8 and 9, fasteners 38 take the form of tacks each having a head 58 and a shaft 60 with one or more backwards extending barbs 62 along shaft 60. Tacks 38 are deployed by ejection mechanism 42 in a circular array about opening 20.

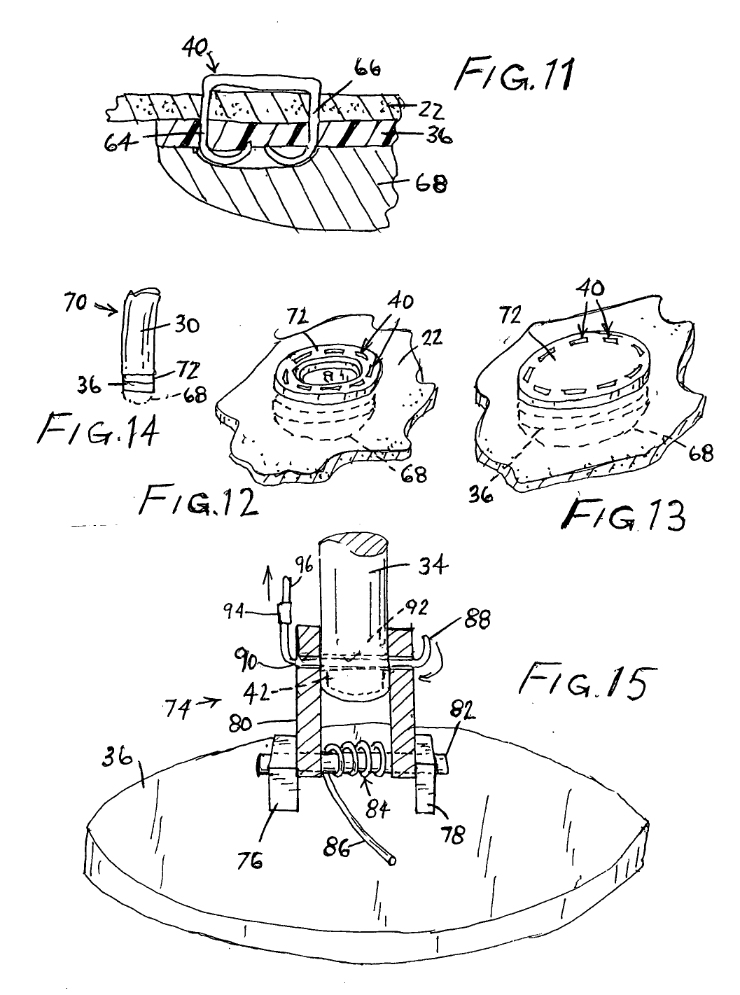

As depicted in FIGS. 10 and 11, fasteners 40 take the form of staples having two legs 64 and 66 that are bent (FIG. 11) upon by an anvil member 68 upon ejection of staples 40 by ejection mechanism 42. Staples 40 are deployed in a circular array about opening 20 (FIG. 10).

In the embodiment of closure device 32 represented by FIGS. 10 and 11, anvil member 68 is attached to disk 36 and remains inside the patient attached to stomach wall 22 at the end of the surgical closure procedure. Thus, anvil member 68 together with disk 36 is attached to rod 34 at the beginning of the procedure and is separated from rod 34 at the end of the procedure.

FIG. 14 illustrates a modified surgical closure device 70 including, in addition to disk 36 and optionally anvil member 68, an ancillary clamping member 72 that is penetrated by fasteners 38 or 40 and connected thereby to disk 36 through stomach wall 22. As shown in FIG. 12, clamping member 72 may take the form of a ring connected to disk 36 about the periphery thereof. Alternatively, as shown in FIG. 13, clamping member 72 may take the form of another disk connected to disk 36 about the periphery thereof.

FIG. 15 shows a coupling assembly 74 whereby disk 36 is pivotably and separably or releasably connected to distal end of rod 34. Disk 36 has a pair of parallel plates 76 and 78 disposed on opposite sides of an auxiliary tube 80 and rotatably attached thereto by a pin 82. A helical spring 84 surrounds pin 82 and has an end 86 engaging disk 36 for angularly biasing disk 36 into a tilted orientation relative to rod 34. Biasing spring 84 tilts disk 36 automatically upon a distal shifting of rod 34 that distances disk 36 from shaft 30. At an end opposite disk 36, auxiliary tube 80 is connected to rod 34 by a flexible pin 88 extending through apertures 90 and 92 in tube 80 and rod 34. Pin 88 is connected at a coupling 94 to a flexible tensile element 96 such as a wire that extends longitudinally along shaft 30 to actuator or control 52.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are profferred by way of example to facilitate comprehension of the invention and should not be construed to limit the scope thereof.

What is claimed is:

1. A surgical closure device comprising:
an elongate tubular shaft;
an elongate rod extending longitudinally through said shaft;
a disk attached to a distal end of said rod;
a plurality of fasteners carried by said shaft at a distal end thereof; and
an ejection mechanism engageable with said fasteners for ejecting said fasteners at least partially through said disk, said disk being separable from said rod.

2. The device defined in claim 1 wherein said fasteners include barbed elements.

3. The device defined in claim 1 wherein said disk is attached on a distal side to an anvil element, said anvil element and said disk being separable as a single unit from said rod, said fasteners including staples.

4. The device defined in claim 1 wherein said fasteners are disposed in a circular array and insert into said disk about an outer edge thereof.

5. The device defined in claim 1 wherein said shaft and said rod are elongate and flexible.

6. The device defined in claim 1, further comprising a ring disposed on said shaft at said distal end thereof proximally of said disk, said ring being attachable to said disk by means of said fasteners.

7. The device defined in claim 1 wherein said disk is a first disk, further comprising a second disk disposed on said shaft at said distal end thereof proximally of said first disk, said second disk being attachable to said first disk by means of said fasteners.

8. The device defined in claim 1 wherein said disk is pivotably attached to said rod.

9. The device defined in claim 1, further comprising means engageable with said disk for tilting said disk relative to said rod so that said disk is disposed at least partially parallel to a distal end portion of said rod.

10. A surgical closure method comprising:
providing a surgical closure device having an elongate tubular shaft, an elongate rod extending longitudinally through said shaft, and a disk attached to a distal end of said rod;
inserting a distal end portion of said shaft into a patient;
thereafter shifting said rod relative to said shaft so that said disk is located distally of a distal end of said shaft;
manipulating said rod to insert said disk through a hole in a wall of a hollow internal organ of the patient;
after the inserting of said disk through said hole, pulling said rod in a proximal direction to bring said disk into contact with said wall;
after said disk is disposed in contact with said wall, ejecting a plurality of fasteners from said distal end of said shaft through said wall and at least partially through said disk while said disk is maintained in contact with said wall; and
after the ejecting of said fasteners, separating said disk from said rod, thereby completing a closure of said hole.

11. The method defined in claim 10 wherein said disk is pivotably attached to said rod, further comprising tilting said disk relative to said rod after the shifting of said rod relative to said shaft and prior to the manipulating of said rod to insert said disk through said hole, said disk being maintained in a tilted orientation relative to said rod during the insertion of said disk through said hole.

12. The method defined in claim 11 wherein said disk is attached on a distal side to an anvil element, said anvil element also being pivotably attached to said rod, the tilting of said disk including a tilting of said anvil element, the inserting of said tilted disk through said hole including inserting the tilted anvil element through said hole, further comprising deforming said fasteners against said anvil during the ejecting of said fasteners from said distal end of said shaft, the separating of said disk from said rod including a separating of said anvil element from said rod, said anvil element and said disk being separated as a single unit from said rod.

13. The method defined in claim 10 wherein the ejecting of said fasteners including ejecting said fasteners in a circular array about an outer edge of said disk.

14. The method defined in claim 10 wherein said shaft and said rod are elongate and flexible, the inserting of a distal end portion of said shaft including moving said distal end portion of said shaft along a curvilinear path.

15. The method defined in claim 10, further comprising placing a ring against said wall after the inserting of said disk through said hole, the placed ring being aligned with an edge of said disk and disposed on a side of said wall opposite the disk, the ejecting of said fasteners including moving said fasteners through said ring, the ejecting of said fasteners resulting in an attachment of said ring to said disk.

16. The method defined in claim 10 wherein said disk is a first disk, further comprising placing a second disk against said wall after the inserting of said disk through said hole, the placed second disk being aligned with an edge of said first disk and disposed on a side of said wall opposite the first disk, the ejecting of said fasteners including moving said fasteners through said second disk, the ejecting of said fasteners resulting in an attachment of said second disk to said first disk.

17. The method defined in claim 10 wherein said disk is pivotably attached to said rod at a center of said disk.
